(19) United States
(12) Patent Application Publication
(10) Pub. No.: US 2008/0303532 A1
(43) Pub. Date: Dec. 11, 2008

(54) COMBUSTIBLE-GAS SENSOR, FUEL CELL SYSTEM, ABNORMALITY DETECTING METHOD, AND FUEL CELL SYSTEM CONTROL METHOD

(75) Inventor: Toru Nakakubo, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/132,795

(22) Filed: Jun. 4, 2008

(30) Foreign Application Priority Data
Jun. 6, 2007 (JP) 2007-149978

(51) Int. Cl. G01R 31/02; G01N 33/497

(52) U.S. Cl. 324/537; 73/23.31

(57) ABSTRACT

A combustible-gas sensor includes a detecting circuit having a bridge comprising a compensator for compensating for a change in external environment and a detector for detecting a concentration of combustible gas. The detecting circuit includes a device for measuring a current passing through the detecting circuit.

### FIELD OF THE INVENTION AND RELATED ART

The present invention relates to a combustible-gas sensor, a fuel cell system including the combustible-gas sensor, an abnormality detecting method of the fuel cell system using the abnormality detecting method.

With advance of development of hydrogen utilization technology such as a fuel cell, a combustible-gas sensor is of increasing importance. Further, development of the fuel cell not only of a stationary type for in-car use or home use but also for mobile devices has been advanced, so that the combustible-gas sensor for these purpose is increasingly required to be reduced in size and electric energy consumption.

Heretofore, combustible-gas sensors of various types have been developed and commercially available. These combustible-gas sensors are roughly classified into three types including an adsorption type, a contact burning (catalytic combustion) type, and a gaseous thermal conduction type and are properly used depending on gaseous species, a detecting area, a response speed, etc. The adsorption type combustible gas is configured to measure a change in electric resistance or volume expansion when gas is adsorbed at a surface of a semiconductor of a metal oxide (such as tin oxide). The contact burning type combustible-gas sensor is configured to measure a change in resistance with temperature rise due to contact combustion of gas at a surface of a catalyst (such as a platinum wire). The gaseous thermal conduction type combustible-gas sensor is configured to measure a change in electric resistance with a change in temperature of a heat generating element (such as a platinum coil) due to a difference in gaseous thermal conductivity.

For example, for detecting leakage of a fuel (hydrogen) in a fuel cell, the contact burning type combustible-gas sensor is frequently used from the viewpoints of a detection concentration and responsivity. A detecting element (detector) of the contact burning type combustible-gas sensor is liable to be affected by an external ambient temperature since it detects a change in resistance with temperature rise due to contact combustion. Therefore, generally, a Wheatstone bridge is formed by using a compensating element (compensator) which has the same size, electric resistance, and heat transfer property as those of the detecting element (detector) but to which a catalyst is not added, thus correcting the influence of the ambient temperature.

These detecting element and compensating element are generally mounted in a suspended state in the air by very thin wires in order to minimize heat transfer with respect to the outside. For that reason, these wires have been broken when they are subjected to impact. When the breaking of wire occurs, a proper output from the sensor, so that leakage of the fuel cannot be properly detected.

In these circumstances, e.g., Japanese Laid-Open Patent Application (JP-A) 2004-127748 has proposed such a system that not only a hydrogen sensor but also an acceleration sensor are provided in a fuel cell system and when rapid acceleration is detected, supply of a fuel is shut off.

Incidentally, with respect to fault of the contact burning type combustible-gas sensor, there is the case where short circuit occurs in addition to the case of the occurrence of the breaking of wire. The short circuit may be attributable to condensation of a sensor module circuit. However, in a conventional combustible-gas sensor driving method in which constant-voltage electric energy is supplied to the sensor, the same output is obtained in both of a combination of breaking of wire of the compensator and short circuit of the detector and a combination of short circuit of the compensator and breaking of wire of the detector, so that it is difficult to discriminate between the breaking of wire and the short circuit. In view of this problem, e.g., JP-A Hei 11-27156 has proposed a method of discriminating between the breaking of wire and the short circuit by measuring a potential between both terminals of the detector or the compensator under a condition that a constant-current power source is used as a power supplying source.

As an energy source to be mounted in a small-size electrical equipment, a small-size fuel cell has received attention. The reason why the fuel cell is useful as a driving source for the small-size electrical equipment may include that it has an amount of supplyable energy, per unit volume or unit weight, which is several to about ten times those of conventional lithium ion secondary batteries. Particularly, for a fuel cell for obtaining a large output, the use of hydrogen as a fuel is most suitable. However, hydrogen is gaseous at normal temperature, so that it requires a technique for storing hydrogen in a small-size fuel tank at a high concentration.

As a method of obtaining hydrogen, there are methods including, other than the use of a high-pressure tank, a method in which hydrogen is extracted from hydrogen-storing alloy or chemical hydride, and a method in which hydrogen is obtained from methanol by reforming. The hydrogen-storing alloy may, e.g., include LaNi₅ and the chemical hydride may, e.g., include sodium borohydride. Further, it is also possible to employ a method in which hydrogen is generated by adding water to metal powder. Generally, a hydrogen dissociation reaction of the hydrogen-storing alloy is endothermic reaction. On the other hand, in the case of the chemical hydride, hydrogen desorption is exothermic reaction in many cases. Further, for the reforming, a reaction temperature is approximately several hundred ° C.

Electric power generation of the fuel cell is accompanied with heat generation. For example, when an energy conversion efficiency in the electric power generation of the fuel cell is 50%, heat is generated in an amount substantially equal to an amount of generated electric power.

In the case of a polymer electrolyte fuel cell (solid polymer fuel cell), in order to promote proton conduction, it is preferable that a polymer electrolyte membrane is wetted. For that reason, a fuel or an oxidizing agent (air) to be supplied is frequently used in a humidified state. Further, also in the case where the humidification is not performed, at a fuel electrode of the fuel cell, water is produced during reaction. For that reason, exhaust air from the fuel cell is higher in temperature and humidity than ambient environment.

Accordingly, when the exhaust air contacts a low temperature portion (such as a wall of a casing), condensation occurs in some cases. Therefore, in the case where the combustible-gas sensor is used in the fuel cell system, it is necessary to particularly pay attention to the condensation.

In the case where the discriminating means for discriminating between the breaking of wire and the short circuit described in JP-A Hei 11-27156 is used, it is impossible to discriminate whether the cause of the short circuit is attributable to fault or condensation. For that reason, irrespective of the case of the short circuit, a system is stopped. However, when supply of electric energy to the gas sensor is stopped in the case where the combustible-gas sensor causes condensation, temperatures of the detector and the compensator are lowered, so that it rather takes a long time to restore the system.

In view of this problem, JP-A 2006-153601 has proposed an apparatus in which a system is stopped in such a manner that the system is judged to be placed in abnormal state when an output value of a gas sensor exceeds a predetermined value but the system is not stopped immediately and only when the abnormal state is continued for a predetermined time or more, the abnormal state is judged to be not attributable to condensation but attributable to short circuit and then the system is stopped.

Further, JP-A 2006-153596 has proposed an apparatus for preventing condensation by providing a heater in the neighborhood of a combustible-gas sensor.

As described above, for detecting leakage of the fuel (hydrogen) of the fuel cell, the contact burning type combustible-gas sensor is suitable from the viewpoints of responsiveness and sensing concentration. However, the contact burning type combustible-gas sensor has such a structure that the detector and the compensator are supported by very thin wires, so that breaking of wire has occurred by impact in some cases.

In such cases, by employing a constitution including an impact sensor (acceleration sensor) as described in JP-A 2004-127748, in the case where a fuel sensor causes breaking of wire or fault due to impact or the like and cannot detect leakage of a fuel, it is possible to stop supply of the fuel before leakage of the fuel occurs.

However, the provision of the impact sensor (acceleration sensor) in addition to the fuel sensor (hydrogen sensor) leads to an increase in cost, so that it was difficult to realize downsizing and cost reduction of the system. Further, in a method of monitoring a device voltage with a contact burning type combustible-gas sensor driven at a constant voltage without using another sensor, it was difficult to discriminate between the breaking of wire and the short circuit.

In the method of monitoring the device current with the sensor driven with the constant current, the power source was complicated to result in increases in size and cost. Particularly, this method was unsuitable for a small-size fuel cell for mobile equipment for driving a sensor with a battery.

In a method in which an output is set to have a threshold as described in JP-A 2006-153601, it was impossible to discriminate between the breaking of wire and the short circuit. Further, in a method of discriminating between condensation and fault depending on a time, it taken a long time to perform the discrimination.

In the case where the sensor causes the breaking of wire due to impact, a considerable impact is exerted on the entire system, so that there is also a possibility of leakage of a fuel. Therefore, during the breaking of wire, it is necessary to immediately stop supply of the fuel but it was difficult to immediately deal with the necessity in the method described in JP-A 2006-153601.

Further, in the method as described in JP-A 2006-153596 as a condensation preventing method, electric power is required for driving the heater, so that there was a possibility of a lowering in system efficiency.

### SUMMARY OF THE INVENTION

A principal object of the present invention is to provide a combustible-gas sensor capable of reliably and quickly stop a system based on judgment made, in a short time, that abnormality is caused due to which of breaking of wire and short circuit without requiring a constant-current power source, thus realizing downsizing of the system.

Another object of the present invention is to provide a fuel cell system including the combustible-gas sensor, a abnormality detecting method of the combustible-gas sensor, and a control method of controlling a fuel cell system using the abnormality detecting method.

According to an aspect of the present invention, there is provided a combustible-gas sensor comprising: a detecting circuit having a bridge comprising a compensator for compensating for a change in external environment and a detector for detecting a concentration of combustible gas, wherein the detecting circuit comprises a device for measuring a current passing through the detecting circuit.

The combustible-gas sensor further comprises an abnormality discriminating device for making a judgment that short circuit occurs when the current passing through the detecting circuit is larger than a predetermined value and that breaking of wire occurs when the current passing through the detecting circuit is smaller than the predetermined value.

According to the present invention, with no need of the constant-current power source, it is possible to judge in a short time that the abnormality is caused due to which of the breaking of wire and the short circuit. Further, it is also possible to realize the combustible-gas sensor capable of making such a judgment, the fuel cell system including the combustible-gas sensor, the abnormality detecting method of the combustible-gas sensor, and the control method of controlling the fuel cell system using the abnormality detecting method.

As a result, the system can be not only reduced in size but also quickly stopped with reliability.

These and other objects, features and advantages of the present invention will become more apparent upon a consideration of the following description of the preferred embodiments of the present invention taken in conjunction with the accompanying drawings.

### BRIEF DESCRIPTION OF THE DRAWINGS

FIG. 1 is a detecting circuit diagram of a combustible-gas sensor in Embodiment 1 of the present invention.

FIG. 2 is a schematic view for illustrating a general arrangement of a combustible-gas sensor (hydrogen sensor) in Embodiment 2 of the present invention.

FIG. 3 is a schematic view for illustrating a general arrangement of a fuel cell system in which the combustible-gas sensor (hydrogen sensor) in Embodiment 2 of the present invention is mounted.

FIG. 4 is a flow chart for illustrating an abnormality detecting method using an abnormality detecting device in Embodiment 2 of the present invention.

FIGS. 5(a) and 5(b) are schematic views each for illustrating a constitutional embodiment of a fuel cell configured to include a heat insulating member for suppressing condensation of the combustible-gas sensor (hydrogen sensor) in Embodiment 2 of the present invention.

FIGS. 6(a) and 6(b) are schematic views each for illustrating a constitutional embodiment of a fuel cell configured to include a heat transfer member for suppressing condensation of the combustible-gas sensor (hydrogen sensor) in Embodiment 2 of the present invention.

FIG. 7 is a schematic view for illustrating a constitutional embodiment of a fuel cell configured to include a water constitute portion for suppressing condensation of the combustible-gas sensor (hydrogen sensor) in Embodiment 2 of the present invention.

FIG. 8 is a schematic view for illustrating a general arrangement of a fuel cell system in Embodiment 3 of the present invention.

FIG. 9 is a flow chart for illustrating an abnormality detecting method using an abnormality detecting device in Embodiment 3 of the present invention.

### DETAILED DESCRIPTION OF THE PREFERRED EMBODIMENTS

The present invention will be described more specifically with reference to the drawings.

### Embodiment 1

In Embodiment 1, a combustible-gas sensor of a contact burning (catalyst combustion) type to which the present invention is applied will be described.

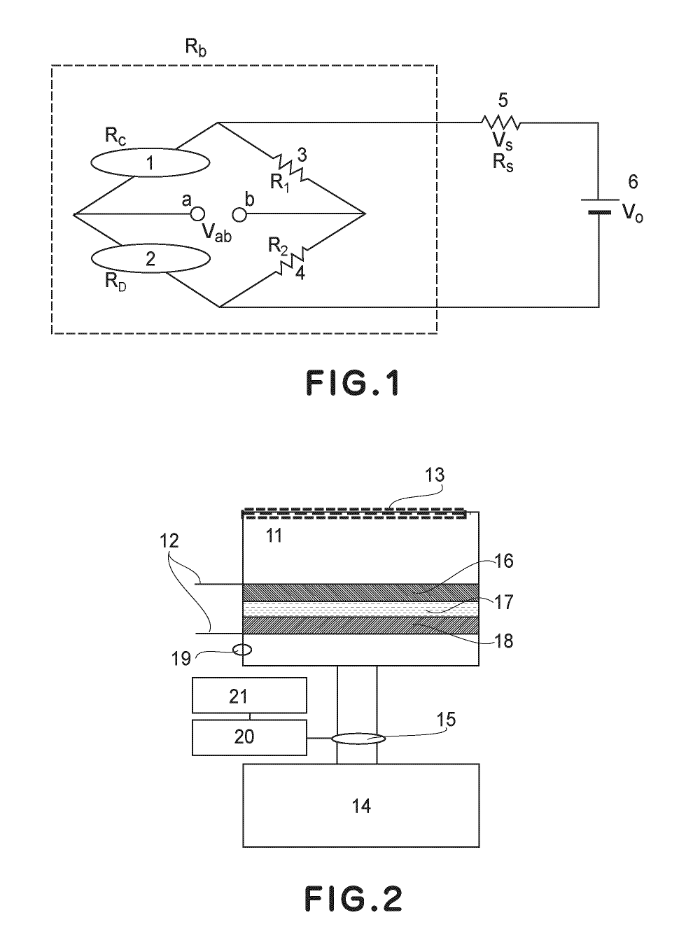

FIG. 1 shows a detecting circuit of the contact burning type combustible-gas sensor in this embodiment.

Referring to FIG. 1, the combustible-gas sensor includes a constitute 1, a detector 2, a first fixed resistance 3, a second fixed resistance 4, an abnormality discriminating resistance 5, and a power source 6.

The combustible-gas sensor in this embodiment is of the contact burning type as described above and is configured to have a detecting circuit constituted by a bridge including the compensator (compensating element) 1 for compensating for a change in external environment and the detector (detecting element) 2 for detecting a concentration of combustible gas. That is, a Wheatstone bridge is formed by the constitute (compensating element) 1 and the detector (detecting element) 2 together with the first resistance 3 (resistance: R₁) and the second fixed resistance 4 (resistance: R₂). Further, it is also possible to add a variable resistance in the circuit in order to finely adjust the circuit. The detecting element is an element which changes its resistance value by heat generated by catalytic combustion of a catalyst for combustion on the element. As the combustion catalyst, it is possible to suitably use palladium, platinum, alloy thereof, and the like. Generally, these catalysts are sintered on a heater coil with alumina or tin oxide as a supporting material but the detecting element used in the present invention is not limited to those including such catalysts. The compensating element has the same structure as that of the detecting element except that it does not include the combustion catalyst. However, in consideration of an ambient temperature characteristic, it is preferable that a copper oxide or the like is added to the supporting material, in place of the combustion catalyst.

The detecting circuit in this embodiment is driven by being supplied with electric energy (power) from a constant-voltage power source 6.

An output of the sensor is obtained by measuring a potential V_{ab} between output terminals a and b.

The detecting circuit in this embodiment includes a device for measuring a current passing through the detecting circuit and this current measuring device is constituted by a fixed resistance placed in series with respect to the bridge and a device for measuring a voltage between both terminals of the fixed resistance. That is, as such a current measuring device, it is possible to employ a constitution in which the abnormality discriminating resistance 5 (resistance: R_{S}) is disposed in series with respect to the Wheatstone bridge and a voltage Vₛ between both terminals of the abnormality discriminating resistance 5 to calculate a current passing through the detecting circuit. For example, electric resistances R_{C} and R_{D} of the compensator 1 and the detector 2 at room temperature and at gas concentration of 0 ppm are taken as 5Ω. Each of the compensator 1 and the detector 2 has a function not only of detecting a signal of the sensor but also as a heater for heating the element itself. In order to keep an end-point temperature of the heater at a constant level, it is preferable that resistances with large resistance values are selected as the first fixed resistance 3 and the second fixed resistance 4 so that a current sufficiently smaller than that passing through the compensator 1 and the detector 2 passes through the resistances 3 and 4. For example, R₁ and R₂ can be 500Ω. In this case, a combined resistance of the bridge is about 9.9Ω as a value obtained through the following equation:

R_{b}=1/(1/(R_{C}+R_{D})+1/(R₁+R₂))

On the other hand, the abnormality discriminating resistance 5 is required to be sufficiently small so as not to largely change a potential exerted on the bridge even when the resistance of the detector and/or the compensator is changed.

However, when R_{S} of the abnormality discriminating resistance 5 is excessively small, it is difficult to detect a potential between the both terminals of the abnormality discriminating resistance 5. For this reason, R_{S} may preferably be about 1/1000 to about 1/50 of the bridge resistance. In this embodiment, R_{S} is 0.1Ω.

When a driving voltage of the sensor is taken as V₀, the voltage V₃ between the both terminals of the abnormality discriminating resistance 5 (R_{S}) is represented by the following equation: V_{S}=V₀R_{S}/(R_{S}+R_{b}).

When the driving voltage V₀ is 2.5 V, the voltage V_{S} is about 25 mV during normal operation.

In the case where at least one of the compensator 1 and the detector 2 causes breaking of wire, V_{S} is 0.25 mV. Further, in the case where the compensator 1 or the detector 2 causes short circuit, V_{S} is 49 mV.

Accordingly, by measuring V_{S}, it is possible to discriminate between the breaking of wire and the short circuit of the sensor. In the case where R_{S} is small and a signal is weak, a signal amplified by an amplifying circuit may also be measured.

In the case where the combustible gas is hydrogen, a lower explosive limit (LEL) of hydrogen is about 4 vol. %. Therefore, it is preferable that a concentration for generating an alarm when hydrogen is leaked as ¼ of the LEL, i.e., about 1 vol. %. The output V_{ab} of the combustible-gas sensor in this embodiment is about 30 mV with hydrogen (0 V, 1%) when the combustible gas is not present.

In the case where the compensator 1 causes the breaking of wire and the detector 2 causes the short circuit, V_{ab}=−V₀R/(R₁+R₂). Further, in the case where the compensator 1 causes the short circuit and the detector 2 causes the breaking of wire, V_{ab}=V₀R(R₁+R₂).

Accordingly, in either of abnormal states (the breaking of wire and the short circuit), the output (voltage) is about ½ of the input voltage, i.e., about ±1.25 V, so that a value largely deviated from an ordinary output value (0 to about 30 mV) is detected.

Thus, the above-described abnormality discriminating can be constituted so as to detect that the output voltage of the combustible-gas sensor exceeds a predetermined range and so as to be actuated or the basis of a result of detected output values. That is, abnormality discriminating is not made in the case where the sensor out is in a normal operation range but is made, only in the case where an abnormal (output) value is detect, as to whether the abnormality is due to the short circuit or the breaking of wire.

By doing so, it is ordinarily not necessary to measure the current passing through the bridge, so that power consumption of the detecting circuit can be reduced. Further, in the case where the sensor output is amplified by the amplifying circuit, the abnormality may also be detected based on the output after the amplification.

The power source used for driving the combustible-gas sensor may preferably be a constant-voltage power source but may also be simply a battery.

In this embodiment, the voltage between the both terminals of the abnormality discriminating resistance 5 is used for detecting the current but another current measuring method may also be employed.

Further, as the discriminating method for discriminating between the breaking of wire and the short circuit, instead of measurement of the current, a resistance of the entire bridge may also be measured. In this case, it is possible to constitute a device for measuring the resistance of the entire bridge by a device for measuring a current passing through the detecting circuit described above and a device for measuring a voltage applied to the both terminals of the bridge.

### Embodiment 2

In Embodiment 2, a constitutional embodiment in which the combustible-gas sensor (hydrogen sensor) of the present invention is mounted in a fuel cell system will be described.

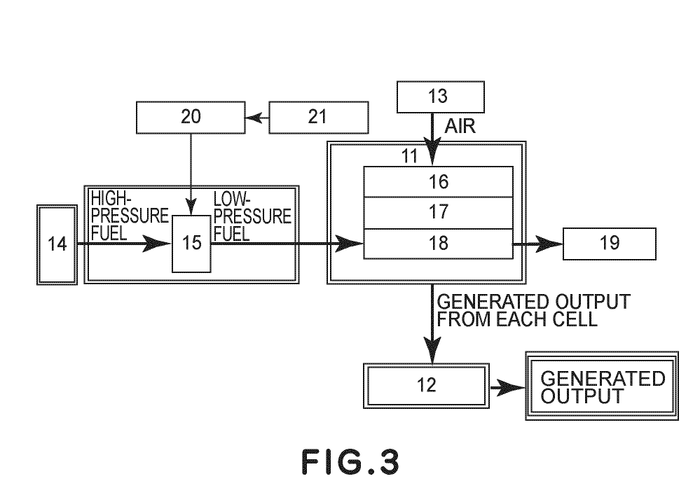

FIG. 2 is a schematic view for illustrating a general arrangement of the combustible-gas sensor (hydrogen sensor) in this embodiment. FIG. 3 is a schematic view for illustrating the constitutional embodiment in which the combustible-gas sensor (hydrogen sensor) in this embodiment is mounted in the fuel cell system.

Referring to FIGS. 2 and 3, the fuel cell system includes a fuel cell 11, an electrode 12, an air bent 13, a fuel tank 14, a fuel supply valve 15, an oxidizing agent electrode 16, a polymer electrolyte film 17, a fuel electrode 18, a purge valve 19, a controller 20, and a combustible-gas sensor (hydrogen sensor) 21.

The fuel cell system described in this embodiment employs hydrogen as a fuel (combustible gas) and air as an oxidizing agent but gases used in the present invention are not limited thereto. Other gases may also be used.

The fuel cell system in this embodiment includes the air bent 13 for taking therein oxygen used for reaction as the oxidizing agent. This air bent 13 also has the function of dissipating generated water as water vapor or dissipating heat generated by the reaction to the outside.

Inside the fuel cell system, the fuel cell 11 consisting of the oxidizing agent electrode 16, the polymer electrolyte film 17 and the fuel electrode 18; the fuel tank 14 for storing the fuel; and the fuel supply valve 15 configured to connect the fuel tank and the fuel electrode of each cell and configured to control a flow rate of the fuel. Further, in side the fuel cell system, the combustible-gas sensor (hydrogen sensor) 21 for detecting leakage of the fuel is provided and based on a signal from the sensor, the fuel supply valve 15 can be controlled by the controller 20.

When the fuel supply valve 15 is opened, hydrogen stored in the fuel tank 14 is supplied to the fuel electrode 18. On the other hand, to the oxidizing agent electrode 16, outside air is supplied. Through the polymer electrolyte film 17 of the fuel cell 11, a catalyst such as platinum is disposed on each of the oxidizing agent electrode 16 and the fuel electrode 18 to cause electrochemical reaction. By this reaction, electric energy is generated and supplied from the electrode 12 to a small-size electric equipment.

In the case where the fuel gas is leaked due to, e.g., connection failure between the fuel tank 14 and the fuel cell 11 or the breaking of wire of the polymer electrolyte film 17, the combustible-gas sensor (hydrogen sensor) 21 detects the leakage. When the detected gas concentration is not less than a predetermined concentration (ordinarily about 1 vol. %), a user is notified of abnormality and the fuel supply valve 15 as a fuel shut-off device is closed to stop generation of electric energy (power).

The combustible-gas sensor (hydrogen sensor) 21 may preferably be located in the neighborhood of a place at which there is a high possibility of the leakage due to the connection failure or the breaking of wire of the polymer electrolyte film 17. Further, in the case where the fuel gas is hydrogen, hydrogen is lighter than air, so that the combustible-gas sensor 21 may preferably be disposed above a casing or in the neighborhood of the air bent 13. The purge valve 19 is configured to perform a purging operation at the time of actuating or stopping the fuel cell or during an operation of the fuel cell. At a discharging portion of the purge valve 19, the combustible-gas sensor (hydrogen sensor) 21 may also be disposed to monitor the concentration of the fuel gas, so that it is possible to control timings of opening/closing of the purge valve 19 and a flow rate of the purge valve 19.

The combustible-gas sensor (hydrogen sensor) 21 is provided with the abnormality detecting device as described in detail in Embodiment 1.

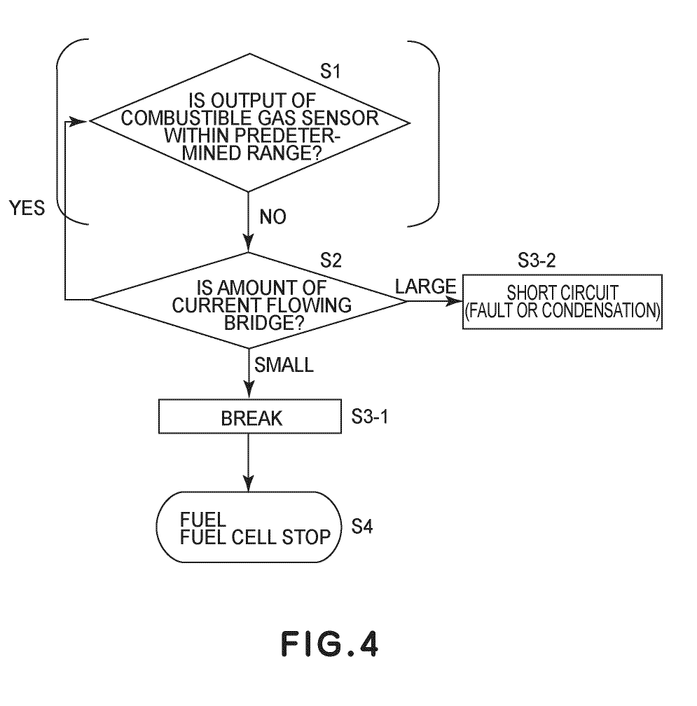

An abnormality detecting method using the abnormality detecting device in this embodiment will be described. FIG. 4 is a flow chart for illustrating the abnormality detecting method in this embodiment.

As shown in FIG. 4, first, a judgment as to whether or not an output of the combustible-gas sensor (hydrogen sensor) 21 is within a predetermined range is made (S1).

In the case where the output if judged to be not within the predetermined range, i.e., the judgment that some abnormality occurs is made (“NO”), in a subsequent step (S2), a judgment as to whether or not an amount of a current passing through the bridge is a normal value is made. In the case where the current passing through the bridge is judged to be small, a judgment that breaking of wire occurs is made (S3-1). Then, a user is notified of the occurrence of the breaking of wire and electric energy generation is stopped by closing the fuel supply valve 15 as a fuel supply control device (S4).

On the other hand, the current passing through the bridge is judged to be large, a judgment that short circuit occurs is made (S3-2) and the user is notified of the occurrence of the short circuit. In this case, the electric energy generation by the fuel cell may be stopped or continued.

Further, similarly as in the case of Embodiment 1, the discrimination of the abnormality may also be performed first in the case where the output of the combustible-gas sensor (hydrogen sensor) 21 is abnormal.

An electric energy generating reaction of the fuel cell is exothermic reaction, so that a temperature of exhaust air from the fuel cell 11 is generally higher than an ambient temperature. Further, the fuel cell is placed in a wet state by generated water. Therefore, when a temperature of the combustible-gas sensor (hydrogen sensor) 21 is lower than the temperature of exhaust air, the sensor is liable to cause condensation. Particularly, in the case where hydrogen-storing alloy is filled and used in the fuel tank 14, a hydrogen dissipation reaction is endothermic reaction. For this reason, a temperature of a surface of the fuel tank 14 is lower than the ambient temperature by about several ° C. Therefore, in the case where the combustible-gas sensor (hydrogen sensor) 21 is disposed in the neighborhood of the fuel tank 14, the condensation of the sensor is liable to occur.

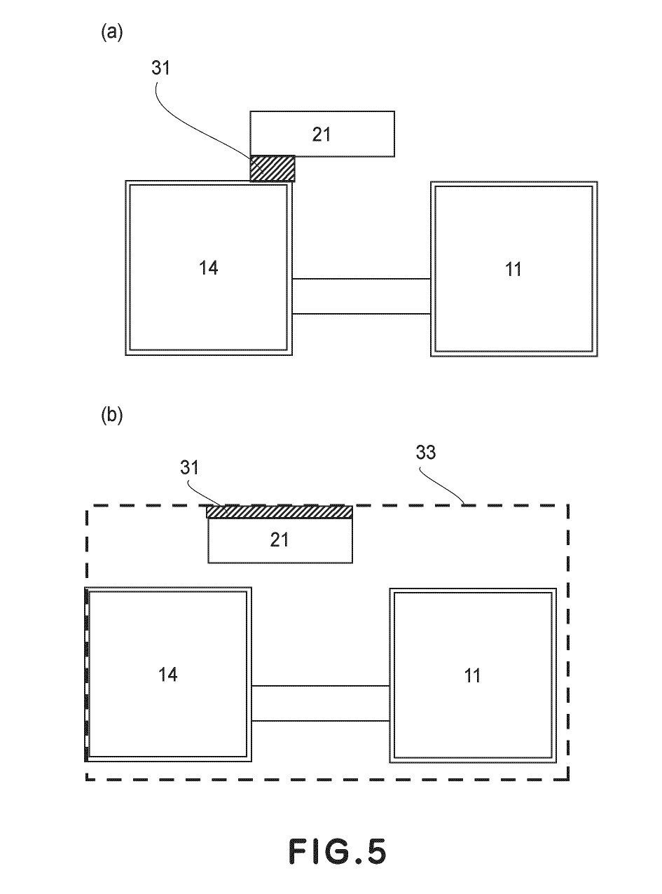

Next, embodiments constituted to suppress the condensation of the sensor will be described with reference to FIGS. 5(a), 5(b), 6(a), 6(b) and 7.

FIGS. 5(a) and 5(b) are schematic views each for illustrating a constitutional embodiment of a fuel cell system configured to include a heat insulating member for suppressing the condensation of the combustible-gas sensor (hydrogen sensor) in this embodiment.

As shown in FIG. 5(a), a heat insulating member 31 is provided between the fuel tank 14 and the combustible-gas sensor (hydrogen sensor) 21, so that the condensation of the combustible-gas sensor 21 is less liable to occur.

Further, as shown in FIG. 5(b), in the case where the fuel tank 14 is thermally connected with a fuel cell casing 33 or the like to lower a temperature of the combustible-gas sensor 21, the heat insulating member 31 may also be disposed between the casing 33 and the sensor 21. By this, the condensation of the combustible-gas sensor 21 is less liable to occur.

In the case where the combustible-gas sensor 21 is disposed at a portion, having a relatively low temperature in the fuel cell system, other than the fuel tank 14, the heat insulating member 31 may preferably be used.

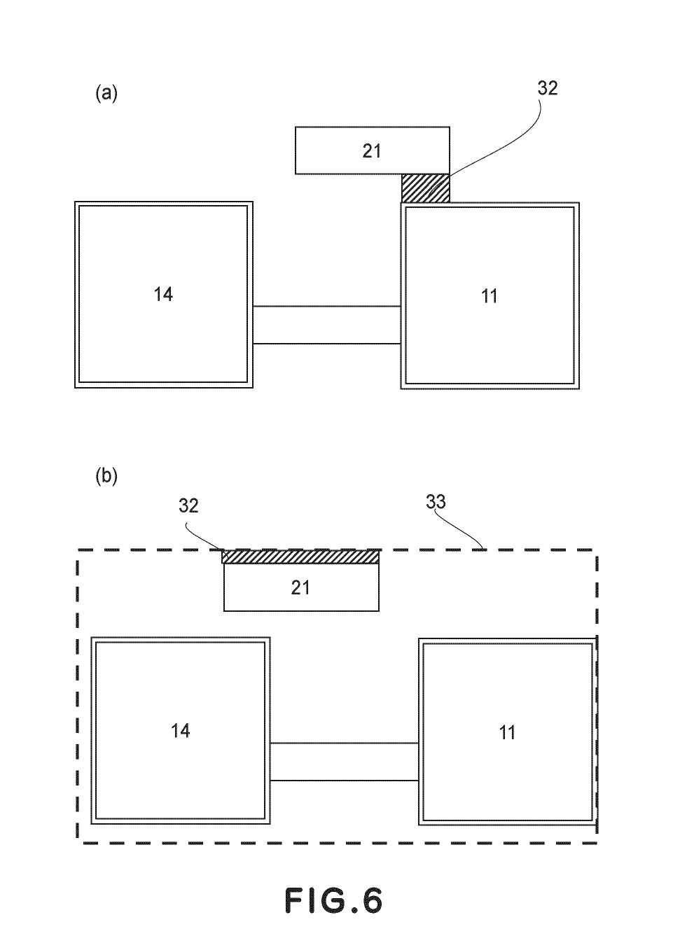

FIGS. 6(a) and 6(b) are schematic views each for illustrating a constitutional embodiment of a fuel cell system configured to include a heat transfer member for suppressing the condensation of the combustible-gas sensor in this embodiment.

In the case where the combustible-gas sensor is provided in the neighborhood of the fuel cell 11, as shown in FIG. 6(a), a heat transfer member 32 may be disposed between the fuel cell 11 and the combustible-gas sensor 21. By this, the temperature of the combustible-gas sensor 21 is increased, so that the condensation of the combustible-gas sensor 21 is less liable to occur. Further, as shown in FIG. 6(b), in the case where the fuel cell 11 is thermally connected with the fuel cell casing 33 or the like, the condensation of the combustible-gas sensor 21 is less liable to occur by providing the heat transfer member 32 between the casing 33 and the sensor 21.

Further, in the case where a material, causing exothermic reaction during supply of hydrogen, such as a chemical hydride is added and used in the fuel tank 14, it is also possible to use the heat transfer member 31 in place of the heat insulating member 32 in FIGS. 5(a) and 5(b). By this, the temperature of the combustible-gas sensor 21 is increased, so that the condensation of the sensor is less liable to occur.

In addition, in the case where the combustible-gas sensor 21 is provided in the neighborhood of a portion having a relatively high temperature in the fuel cell system, it is preferable that the heat transfer member 32 is used.

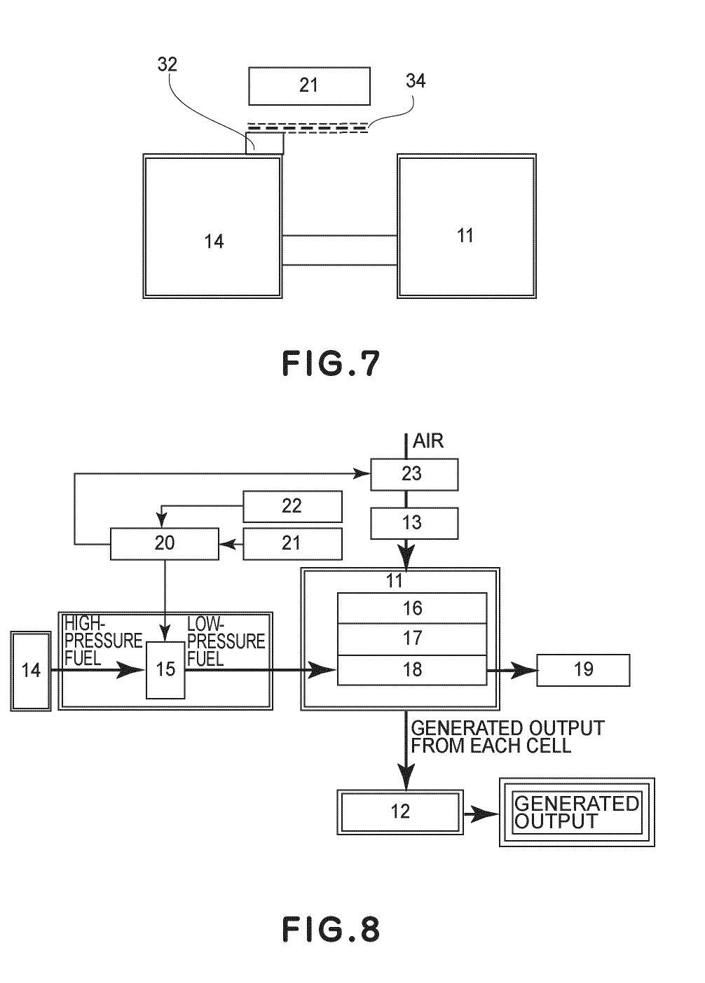

FIG. 7 is a schematic view for illustrating a constitutional embodiment of a fuel cell system configured to include a water condensation portion for suppressing the condensation of the combustible-gas sensor. As a method of preventing the condensation of the combustible-gas sensor 21, it is possible to employ such a constitution that the temperature is lowered before the gas contacts the sensor to condense water. For example, as shown in FIG. 7, a water condensation portion 34 can be disposed between the combustible-gas sensor 21 and a flow path. Further, in order to lower the temperature of the water condensation portion 34, between the water condensation portion and the portion having a relatively low temperature in the fuel cell system, the heat transfer member 32 may be disposed. The portion having a relatively low temperature may, e.g., include a fuel tank 14 in the case of containing the hydrogen-storing alloy.

The combustible-gas sensor 21 is less affected by the ambient temperature by providing the compensator 1 but it is difficult to completely eliminate the influence of the ambient temperature. Therefore, the combustible-gas sensor 21 may also be configured depending on a temperature at which the fuel cell system is subjected to rated operation. Further, it is also possible to improve precision of measurement of a combustible-gas concentration by using different calibrated values for the time of fuel cell stop (or during low-output electric energy generation) and the time of rated generation of electric energy (during high-output electric energy generation).

### Embodiment 3

In FIG. 3, a constitutional embodiment in which a fuel cell system including the fuel cell system in Embodiment 2, a humidity sensor for detecting a humidity in the system, and a fan for supplying a larger amount of air and discharging water vapor will be described.

FIG. 8 is a schematic view for illustrating a general arrangement of the fuel cell system in this embodiment in which the combustible-gas sensor (hydrogen sensor) is mounted.

In FIG. 8, members or means common to those used in Embodiment 2 are represented by reference numerals identical to those indicated in FIG. 3. A constitution of this embodiment common to that of Embodiment 2 will be omitted from the following description.

Referring to FIG. 8, the fuel cell system in this embodiment includes a humidity sensor 22 and a fan 23.

In this embodiment, an operation of a fuel cell in an ordinary (normal) state is similar to that in Embodiment 2. However, in the case where a judgment that a humidity is high is made by the humidity sensor 22, the volume of air of the fan 23 is increased. On the other hand, in the case where a judgment that the humidity is low is made by the humidity sensor 22, the volume of air of the fan 23 is decreased. By controlling the fan 23 in this manner, stability of the system is improved.

Next, an abnormality detecting method by an abnormality detecting device in this embodiment will be described.

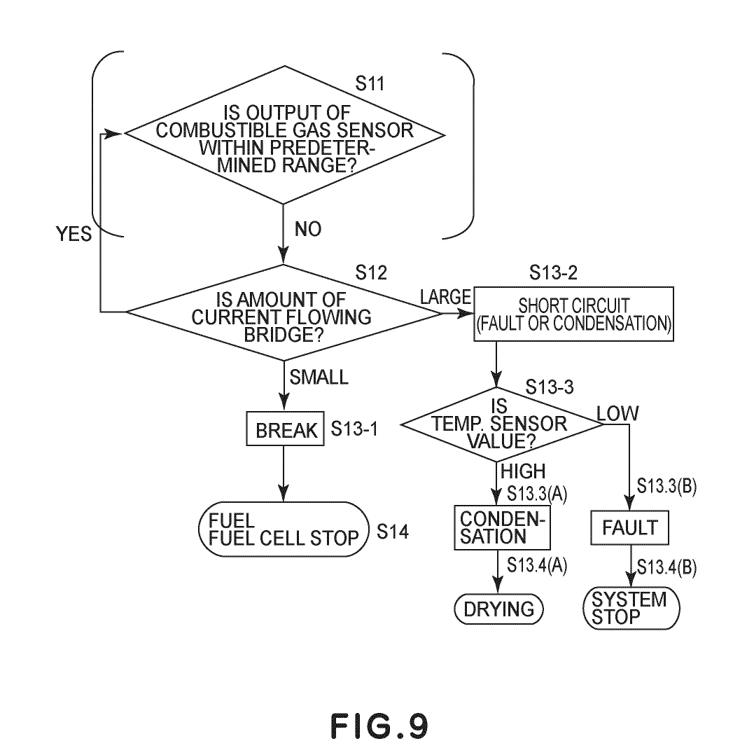

FIG. 9 is a flow chart for illustrating the abnormality detecting method by the abnormality detecting device in this embodiment.

As shown in FIG. 9, first, a judgment that an output of the combustible-gas sensor (hydrogen sensor) 21 is within a predetermined range is made (S11).

During detection of abnormality of the combustible-gas sensor 21 (S12), in the case where an amount of a current passing through the bridge is large, the abnormality is judged to be caused due to short circuit (S13-2). Then, with reference to a value of the humidity sensor 22 (S13-3), when an ambient humidity is high, the short circuit is judged to be caused due to condensation (S13.3 (A)). In this case, a user is notified of an occurrence of the condensation and the system is dried by the fan 23 so as to be readily restored from a condensation state to the normal state (S13.4 (A)). In the case where the short circuit is judged not to be caused due to the condensation, the system is judged to cause fault (S13.3 (B)). Then, the user is notified of an occurrence of the fault and the operation of the system is stopped (S13.4 (B)).

On the basis of a signal of the humidity sensor 22, when the humidity is increased before the combustible-gas sensor 21 causes the condensation, the system may be dried by increasing the amount of volume of air of the fan 23 to obviate the condensation state.

During detection of the abnormality of the combustible-gas sensor 21 (S12), in the case where the amount of the current passing through the bridge is small, the abnormality is judged to be caused due to breaking of wire (S13-1). Then, the user is notified of an occurrence of the breaking of wire and the fuel supply valve 15 as a fuel supply control device is closed thereby to stop generation of electric energy (S14).

In the case where the fuel cell system does not include the humidity sensor 22, it is also possible to monitor the humidity by abnormality detection with the combustible-gas sensor 21. That is, in the case where the combustible-gas sensor 21 is judged to cause the short circuit, a judgment that the condensation occurs is made, so that it is possible to obviate an occurrence of blockage of a flow path of the fuel cell 11 due to the condensation (flooding) by, e.g., increasing the amount of volume of air of the fan 23.

As described in detail above, the present invention relates to abnormality detection of a combustible-gas sensor module of the type wherein a bridge is created by a constitute and a detector to provide an output in order to detect leakage of a fuel.

An output value generated during fault of the combustible-gas sensor is stored and in the case where the output value is detected, the combustible-gas sensor is judged to be placed in an abnormal state (breaking of wire or short circuit). Further, by use of a device for measuring a current passing through a circuit of the combustible-gas sensor module or measuring a resistance of the bridge, a judgment as to whether the abnormality is the breaking of wire or the short circuit is made on the basis of the detected current value. Particularly, in the fuel cell system including the combustible-gas abnormality detecting device in the present invention, in the case where the abnormality is judged to be caused due to the breaking of wire, the fuel cell system is stopped immediately.

Further, by comparison with a signal of the humidity sensor in the fuel cell system, discrimination between the short circuit and condensation is performed when the short circuit of the sensor occurs and restoring of the system from the condensation state is performed by controlling the fan.

As a result, during an occurrence of the abnormality of the combustible-gas sensor, it is possible to judge in a short time as to whether the abnormality is caused due to the breaking of wire or the short circuit. Further, in the case where, e.g., such an impact that the fuel is leaked is exerted on the fuel cell system, it is possible to stop the fuel cell system immediately to stop supply of the fuel.

While the invention has been described with reference to the structures disclosed herein, it is not confined to the details set forth and this application is intended to cover such modifications or changes as may come within the purpose of the improvements or the scope of the following claims.

This application claims priority from Japanese Patent Application No. 149978/2007 filed Jun. 6, 2007, which is hereby incorporated by reference.

What is claimed is:

1. A combustible-gas sensor comprising:
a detecting circuit having a bridge comprising a compensator for compensating for a change in external environment and a detector for detecting a concentration of combustible gas,
wherein said detecting circuit comprises a device for measuring a current passing through said detecting circuit.

2. A sensor according to claim 1, wherein said combustible-gas sensor further comprises an abnormality discriminating device for making a judgment that short circuit occurs when the current passing through the detecting circuit is larger than a predetermined value and that breaking of wire occurs when the current passing through the detecting circuit is smaller than the predetermined value.

3. A sensor according to claim 2, wherein the abnormality discriminating device is configured to be actuated on the basis of detection of an output by a device for detecting that an output value of said combustible-gas sensor exceeds a predetermined range.

4. A sensor according to claim 1, wherein said detecting circuit further comprises a device, for measuring a resistance of the bridge, comprising the device for measuring the current passing through the detecting circuit and a device for measuring a potential difference between both ends of the bridge.

5. A fuel cell system comprising:
a combustible-gas sensor according to claim 1;
a fuel cell; and
a fuel tank.

6. A system according to claim 5, wherein said fuel cell system further comprises a fuel shutoff device,
wherein said combustible-gas sensor further comprises an abnormality discriminating device for making a judgment that breaking of wire occurs when the current passing through the detecting circuit is smaller than the predetermined value, and
wherein said fuel shutoff device is configured to shut off supply of fuel gas on the basis of the judgment by said abnormality discriminating device.

7. A system according to claim 5, wherein said fuel cell system further comprises a humidity detecting device,
wherein said combustible-gas sensor further comprises an abnormality discriminating device for making a judgment that short circuit occurs when the current passing through the detecting circuit is larger than a predetermined value, and
wherein said humidity detecting device is configured to judge whether or not short circuit is caused due to condensation on the basis of a value detected by the humidity detecting device in the case where the abnormality discriminating device makes judgment that the short circuit occurs.

8. A system according to claim 5, wherein said fuel cell system further comprises a heat transfer member disposed between said combustible-gas sensor and a portion relatively higher in temperature in said fuel cell system.

9. A system according to claim 8, wherein the portion relatively higher in temperature is said fuel cell.

10. A system according to claim 8, wherein the portion relatively higher in temperature in said fuel tank and wherein said fuel tank is increased in temperature with supply of fuel.

11. A system according to claim 5, wherein said fuel cell system further comprises a heat insulating member disposed between said combustible-gas sensor and a portion relatively lower in temperature in said fuel cell system.

12. A system according to claim 11, wherein the portion relatively lower in temperature in said fuel tank and wherein said fuel tank is decreased in temperature with supply of fuel.

13. A system according to claim 5, wherein said fuel cell system further comprises a water condensation portion disposed between said combustible-gas sensor and a portion relatively lower in temperature in said fuel cell system and wherein said water condensation portion is connected to the portion relatively lower in temperature through a heat transfer member.

14. An abnormality detecting method for detecting abnormality of a combustible-gas sensor, comprising:
providing a detecting circuit having a bridge comprising a compensator for compensating for a change in external environment and a detector for detecting a concentration of combustible gas, and a current measuring device for measuring a current passing through the detecting circuit; and
measuring the current passing through the detecting circuit by the current measuring device to detect abnormality of the combustible-gas sensor.

15. A method of according to claim 14, wherein a voltage between both terminals of a fixed resistance is measured to make judgment that short circuit occurs when the current passing through the detecting circuit is larger than a predetermined value and that breaking of wire occurs when the current passing through the detecting circuit is smaller than the predetermined value.

16. A method according to claim 15, wherein the measurement of the current passing through the detecting circuit is performed when an output value of said combustible-gas sensor exceeds a predetermined range.

17. A method according to claim 14, wherein said detecting circuit is configured to include a device for measuring a resistance of the bridge, and wherein the current passing through the detecting circuit and a device for measuring a potential difference between both ends of the bridge are measured by the device for measuring the resistance of the bridge.

18. A control method of controlling a fuel cell system comprising a combustible-gas sensor according to claim 14, a fuel cell, and a fuel tank, said control method comprising:
detecting abnormality of the combustible-gas sensor.

19. A method according to claim 18, wherein the fuel cell system further comprises a fuel supply control device for controlling supply of a fuel from the fuel tank to the fuel cell, and wherein the fuel supply control device shuts off the supply of the fuel when breaking of wire is detected by the combustible-gas sensor.

20. A method according to claim 18, wherein said fuel cell system further comprises a humidity detecting device, and wherein when short circuit is detected by the combustible-gas sensor, a judgment as to whether or not the short circuit is caused due to condensation is made on the basis of a value of the humidity detecting device.
